(19) United States
(12) Patent Application Publication
(10) Pub. No.: US 2006/0162074 A1
(43) Pub. Date: Jul. 27, 2006

(54) Device and method for controlling physical properties of a bed

(76) Inventor: Gaby Bader, Goteborg (SE)

Correspondence Address: HARNESS, DICKEY & PIERCE, P.L.C., P.O. BOX 8910, RESTON, VA, 20195, US

(21) Appl. No.: 10/544,380

(22) PCT Filed: Feb. 2, 2004
(86) PCT No.: PCT/SE04/00135

§ 371 Date: Aug. 3, 2005

(30) Foreign Application Priority Data
Feb. 4, 2003 (SE) 0300280-5

(51) Int. Cl. A47C 21/04; A61B 5/02; A61B 5/00; A61B 5/08; A61B 5/103; A61F 7/00

(52) U.S. Cl. 5/421; 600/549; 600/587; 600/595; 600/529; 600/508

(57) ABSTRACT

A system and a method are proposed for controlling at least one physical property of a bed based on the state of a person staying in the bed. A lying part in the bed is provided with a plurality of sensors adapted to continuously measure physical magnitudes as a measurement of the person's state. Measured values from the sensors are received, processed and analysed by a registration unit, for controlling physical properties of the bed.

### TECHNICAL FIELD

The present invention relates to a device and a method for controlling physical properties of a bed with a person in it.

### BACKGROUND ART

In sleep, the bed used is frequently not optimally adapted to the sleeping person's state and constitution. After falling asleep, it may happen that the person wakes up after some time owing to an increasing stiffness, and also backache may arise. It is a well-known condition that, when buying a bed, it should be carefully tested to find out the optimal resilience and hardness, adapted to the individual's weight and shape. However, the need for different properties of the bed changes over time, both in the long run and during the night. Moreover, the sleep rhythm may vary to a great extent, individually as well as between different occasions, with regard to periods of deep sleep, dream activity, snoring etc.

Difficulties in falling asleep and sleep troubles can to some extent be attended to by adaptation of temperature, ventilation, and also by variation of lying posture. However, such measures require efforts to be made by the person, which of course interferes with the process of falling asleep and the sleep. One and the same person may thus have a highly varying need for heat/cold during the time he/she is in the bed. For example, it may be desirable to go to sleep in a relatively warm bed at night, while coolness is required during sleep and possibly when waking up. Perspiration is common during sleep and when waking up, for instance due to dreams or fever.

It is thus desirable to optimise comfort for a person being in a bed. It is difficult, for instance, to maintain an appropriate temperature and hardness which is comfortable for a person lying in a bed for the entire duration of staying therein. When confined to bed for a long time, injuries or bedsore may arise in people who for some reason are relatively immobile owing to, for instance, bodily injury, sickness, disablement or old age. It may also be uncomfortable in general to lie still for long periods, especially in the same posture, which often results in stiffness and numbness. There is thus a need to be able to control different properties of a bed with a person in it, with a view to providing great comfort and preventing unpleasantness and injuries. It is also desirable to be able to control the environment in the room where an individual is sleeping.

Another interesting field involves brief interruptions of breathing caused by snoring, which is referred to as sleep apnea, which may cause unsatisfactory sleep quality and, if the worst comes to the worst, brain damage. Of course, snoring also disturbs other people. Several solutions to this problem have been developed, such as surgery or appliances that fit in the mouth to prevent snoring. However, such measures are of course embarrassing to the person. A significantly simpler method is to sleep in a more or less upright position, which tends to prevent snoring. Such a sleeping position, however, is uncomfortable in the long run, and therefore the sleeping person often changes his posture during sleep and resumes snoring.

### DESCRIPTION OF THE INVENTION

An object of the present invention is to provide great comfort in a bed for a person staying in the bed. Another object is to prevent snoring and interruptions of breathing during sleep caused by snoring. Another object is to provide good hygiene in a bed.

These and other objects are achieved by a system and a method for controlling at least one physical property of a bed based on the state of a person staying in the bed, according to the appended claims. The inventive system comprises a lying part to be included in a bed and provided with a plurality of sensors adapted to continuously measure physical magnitudes as a measure of the person's state. The system also comprises a registration unit for registering measured values from the sensors.

### BRIEF DESCRIPTION OF THE DRAWING

The present invention will now be described in more detail by way of a preferred embodiment and with reference to the accompanying drawing, in which

FIG. 1 is a schematic perspective view of a system for controlling at least one physical property of a bed.

### DETAILED DESCRIPTION OF EMBODIMENTS

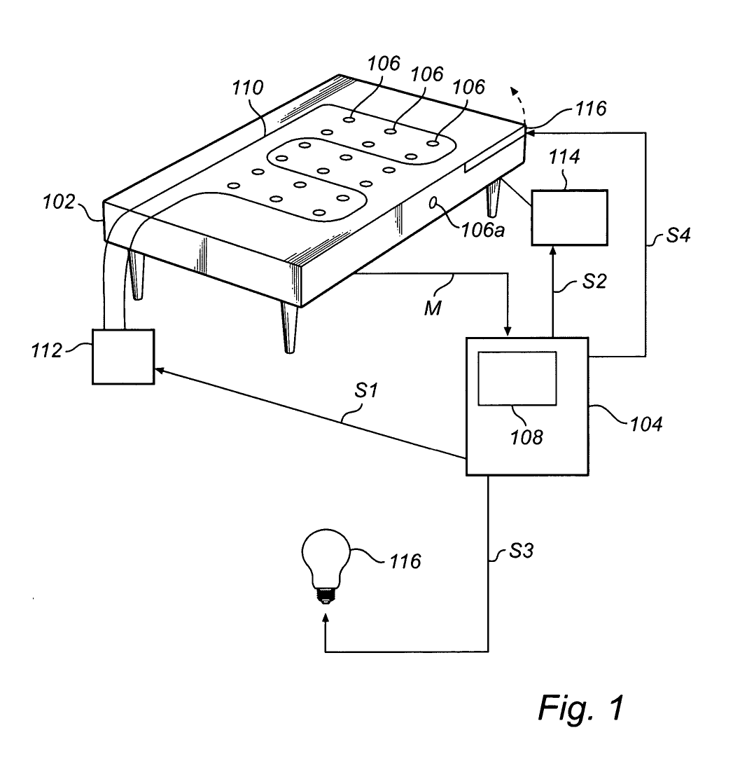

FIG. 1 is a schematic view of a system 100 for controlling at least one physical property of a bed and/or the surrounding environment with a person staying in the bed. The system comprises a lying part 102 which is included in a bed, and a registration unit 104. The lying part can be a mattress or the like which is formed either integrally with the bed or as a separate part to be placed on the bed. The lying part 102 is provided with a plurality of sensors 106 for measuring one or more physical magnitudes with a person staying in the bed. The sensors 106 can be adapted to measure one or more of the following physical magnitudes: temperature, pressure, movements, sound and moisture. The system 100 may further comprise one or more sensors 106a arranged outside the lying part, such as at the side of the bed or in some other convenient place, for instance for measuring sound and air temperature.

Moreover a measured physical magnitude may constitute an indirect measurement of another magnitude. For example, the measured temperature in the lying part 102 can be used to analyse pressure distribution on the same when a person is staying in the bed. An increased temperature at a certain point or in a small area of the lying part for a certain time can be interpreted as an indication of an increased pressure compared with the remaining surface.

The registration unit 104 comprises a programmable processor 108 for receiving and processing measured values M from the sensors 106. Thus the registration unit 104 is adapted to receive, process and analyse measured values obtained from the sensors 106 in order to obtain information about the person. For example, different parameters can be calculated, describing the distribution of the magnitude over the surface covered by the sensors, i.e. spatial information, and how the magnitude has changed during a period of time, i.e. temporal information. According to the present invention, such information is used to control at least one physical property of the bed and/or the surrounding environment.

It should be noted that the present invention is not restricted to a special arrangement or special components in order to convey measured values of physical magnitudes from the sensors to the registration unit.

The following information about the person can thus be provided by means of measured values from the sensors:

The person's movements, such as turning over in the bed.

Sound, such as detection of snoring.

The person's breathing activity that can be registered through movements and/or sound.

The person's heart activity which can also be registered through movements and/or sound.

Temperature of the person and of the air.

Pressure exerted on the bed's lying surface, which indirectly can be registered through temperature.

Moisture of the bed and of the air.

The person's posture relative to the lying surface.

The registration unit 104 can be programmed to process received measured values in order to perform a more or less continuous analysis of the person's state, for instance with regard to wakefulness, sleep depth, dream activity and phase in the sleep cycle. This information can then be used to optimise comfort and sleep quality by one or more measures being taken. Below follow some relatively simple examples of such measures:
1. The temperature of the bed can be controlled according to the measured temperature of the bed. To this end, the bed is provided with a temperature coil 110 or the like, adapted to be controlled by a temperature control unit 112. The temperature coil 110 can thus be adapted to heat and/or cool the lying part, for instance by means of an electric coil or a suitable flowing medium. When one or more sensors 106 indicate that the temperature of the bed, for instance, exceeds a predetermined level for a certain time, the registration unit 104 emits a control signal S1 to the temperature control unit 112, which then activates the temperature coil 110 so that the temperature is lowered in the bed to a desirable level. Moreover, the registration unit 104 can be programmed so that different temperatures are set according to hour or point of time during a sleep cycle, or if perspiration is detected. For example, a relatively high temperature can be desirable when falling asleep, while a lower level is more convenient at a later stage.
2. The hardness of the lying surface can be adapted to the person's weight, state and/or point of time. To this end, the bed is provided with a mechanism for adjusting the bed firmness, not shown, which is adapted to be controlled by a firmness control unit 114. When, for instance, the person goes to bed, a relatively soft bed can be desirable, whereas a harder bed is more convenient during sleep. When one or more sensors 106 indicate that the person falls asleep, the registration unit 104 emits a control signal S2 to the firmness control unit 114, which then ensures that the hardness of the lying surface is changed from being soft to become harder. In connection with waking up, a new control signal is emitted so that the lying surface again becomes soft, which means that the person falls asleep and wakes up in a soft bed.
   The bed firmness can also be changed locally at a point or in a limited area of the lying surface. A raised temperature at a certain point or in a small area of the lying surface during a certain time may be an indication of an increased pressure compared with the remaining surface. Alternatively, the pressure can, of course, also be measured directly using pressure sensors. Such an increased pressure may cause discomfort for the person, such as stiffness and numbness and, in unfavourable cases, bedsore. This information can then be used to change the bed firmness locally so as to relieve the pressure at this point. This application is above all useful for people who are motor-handicapped owing to, for instance, sickness, old age, paralysis or injury, but also to increase the lying comfort in general. Such information about pressure can also be used in order to optimally adapt the hardness of the bed to the person's weight.
3. When a current sleep analysis indicates that the person is about to wake up, after a suitable point of time, for instance, in the morning, the light can be turned on, preferably by gradually increasing the illumination in order to simulate sunrise. To this end, the registration unit 104 is connected to a suitable light source 116, to which a control signal S3 is sent.
4. The registration unit 104 can be programmed to detect if the person snores and optionally has interruptions of breathing caused by snoring, so-called sleep apnea. A programmed suitable algorithm can then be used to detect if significant interruptions of breathing occur while the person is sleeping, by registration of the number and duration of the interruptions, and if they have been going on for a predetermined time. When such significant interruptions of breathing are detected, or when long snoring periods are detected, for instance, by registration of sound, a control signal S4 is emitted, which activates a vertically adjustable part 116 positioned at the head end of the bed, which is indicated by a dashed arrow in the FIGURE, so that the upper part of the person's body is raised so as to reduce his tendency to snore. If snoring/interruption of breathing remains, the vertically adjustable part 116 can be further raised. After a predetermined period of time, and before waking up, a new control signal can be emitted so that the vertically adjustable part 116 is again lowered to a flat position so that the person will wake up in a lying position.
5. Occasional sleep difficulties can be detected by measured values from suitable sensors, which for instance indicate that the person is moving around a lot. To facilitate falling asleep, the sleep environment can then be changed, for instance by changing the temperature of air, airing or activating relaxation sound.
6. On detection that the person has left the bed, airing of the bed can be carried out automatically by injecting air into the mattress, for drying bedclothes and removing mite allergens. For instance, a compressor can be automatically operated for injecting air into the mattress, possibly mixed with, for instance, microdroplets of detergents or antiseptics, through small flexible tubes integrated into the mattress (not shown).

The above described sensors 106, 106a can be connected individually or in groups to a registration unit 104 by cables 106A-C, or be equipped for wireless communication, for registering measured values from the sensors. When connected in groups, each group of sensors and the associated cable connection can form a measuring coil with sensors connected in series, so that measured values from the individual sensors in the group can be read sequentially, which in itself constitutes prior-art technique in the field of measuring.

The present invention provides a smooth and reliable solution to obtain optimal comfort and sleep quality in a bed with a person in it, without troubling the person in any way.

The invention is not restricted to the above embodiments and is defined in general by the appended claims.

1. A system for controlling at least one physical property of a bed with a person in it, comprising:
a lying part included in the bed and provided with a plurality of sensors for measuring one or more physical magnitudes; and
a registration unit adapted to receive, process and analyse measured values from the sensors, and control at least one physical property of the bed based on measured values received from the sensors the registration unit is further adapted to detect if the person snores and his breathing is possibly interrupted by snoring, and to activate a vertically adjustable part positioned at the head end of the bed so that the upper part of the body of the person is raised when significant interruptions of breathing are detected or when long snoring periods are detected.

2. A system as claimed in claim 1, wherein the sensors are adapted to measure one or more of the following physical magnitudes: temperature, pressure, movements, sound and moisture.

3. A system as claimed in claim 1, wherein one or more sensors are arranged outside the lying part.

4. A system as claimed in claim 1, wherein the registration unit comprises a programmable processor for receiving and processing measured values from the sensors.

5. A system as claimed in claim 1, wherein the registration unit is adapted to produce, using measured values from the sensors, the following information: movements, sound, breathing activity, heart activity, temperature of the person and of the air, pressure exerted on the lying surface, moisture of the bed and of the air, and the person's posture relative to the lying surface.

6. A system as claimed in claim 1, further comprising a temperature control unit for controlling the temperature of the bed according to at least one of the following: measured temperature of the bed, hour, point of time in a sleep cycle, and if perspirations are detected.

7. A system as claimed in claim 1, further comprising a firmness control unit for controlling a mechanism for adjusting the firmness of the bed, so that the hardness of the lying surface is adapted to at least one of the person's weight, state and point of time.

8. A system as claimed in claim 7, wherein the firmness control unit is adapted to change the firmness of the bed locally at a point or in a limited area of the lying surface.

9. A system as claimed in claim 1, wherein the registration unit is connected to a light source in order to turn on the light when the current sleep analysis indicates that the person is about to wake up, after a suitable point of time.

10. A system as claimed in claim 1, wherein the system is adapted to change the sleep environment in order to facilitate the person's falling asleep when occasional sleep difficulties are detected.

11. A system as claimed in claim 1, wherein the system is adapted to automatically perform airing of the bed by injecting air into the mattress of the bed when it has been detected that the person leaves the bed.

12. A method for controlling at least one physical property of a bed with a person in it, comprising:
measuring one or more physical magnitudes by a plurality of sensors positioned in a lying part included in the bed;
receiving, processing and analysing measured values from the sensors; and
controlling at least one physical property of the bed based on measured values received from the sensors.

13. A method as claimed in claim 12, wherein the measuring includes measuring at least one of the following physical magnitudes: temperature, pressure, movements, sound and moisture, and the controlling includes
controlling at least one of the following physical properties: temperature, hardness of the lying surface and inclination of a vertically adjustable part located at the head end of the bed.

14. A system as claimed in claim 2, wherein one or more sensors are arranged outside the lying part.

15. A system as claimed in claim 2, further comprising a temperature control unit for controlling the temperature of the bed according to at least one of the following: measured temperature of the bed, hour, point of time in a sleep cycle, and if perspirations are detected.

16. A system as claimed in claim 2, further comprising a firmness control unit for controlling a mechanism for adjusting the firmness of the bed, so that the hardness of the lying surface is adapted to at least one of the person's weight, state and point of time.

17. A system as claimed in claim 16, wherein the firmness control unit is adapted to change the firmness of the bed locally at a point or in a limited area of the lying surface.

18. A system as claimed in claim 2, wherein the registration unit is connected to a light source in order to turn on the light when the current sleep analysis indicates that the person is about to wake up, after a suitable point of time.

19. A system as claimed in claim 2, wherein the system is adapted to change the sleep environment in order to facilitate the person's falling asleep when occasional sleep difficulties are detected.

20. A system as claimed in claim 2, wherein the system is adapted to automatically perform airing of the bed by injecting air into the mattress of the bed when it has been detected that the person leaves the bed.
